(19) United States
(12) Patent Application Publication
(10) Pub. No.: US 2006/0292702 A1
(43) Pub. Date: Dec. 28, 2006

(54) Method for modeling an simulating a biological system, and model carrying out this method

(76) Inventors: Magali Rouquie, Paris (FR); Nicolas Caritey, St. Hilaire (FR); Laurent Gaubert, Marseille (FR)

Correspondence Address: MILLER, MATTHIS & HULL, ONE NORTH FRANKLIN STREET, SUITE 2350, CHICAGO, IL, 60606, US

(21) Appl. No.: 10/568,718

(22) PCT Filed: Aug. 10, 2004
(86) PCT No.: PCT/FR04/02115

§ 371 Date: Jun. 14, 2006

(30) Foreign Application Priority Data
Aug. 18, 2003 (FR) 0309983

(51) Int. Cl. G06F 19/00; G01N 33/543

(52) U.S. Cl. 436/518; 702/19

(57) ABSTRACT

A method of modeling and simulating a biological system comprising one or more tangible biological functional entities modeled by at least: a morphological occurrence comprising at least a biochemical constituent that identifies the biological functional entity and at least a transformation representing the way in which that constituent behaves as a function of the space-time context, a spatial occurrence representing at least a spatial characteristic of the biological functional entity, and a temporal occurrence representing at least a temporal characteristic of the biological functional entity, which method simulates the behavior of said biological functional entities by recursively determining the effect on their functioning and on their behavior (their activities) of all changes affecting said occurrences, including transformations.

### FIELD OF THE INVENTION

The present invention relates to methods of modeling and simulating biological systems.

### BACKGROUND OF THE INVENTION

Methods of this kind have been described by ROUX-ROUQUIE et al. (R. C. Biologies 325 (2002) 419-430), for example.

Prior art methods of modeling and simulating biological systems have the drawback of being implemented on an ad hoc basis and in a non-standardized manner, with the result that they are incompatible with each other and difficult to understand for biologists who are not familiar with applied mathematics. They are therefore difficult to validate and of uncertain reliability.

### SUMMARY OF THE INVENTION

One particular object of the present invention is to alleviate these drawbacks.

To this end, the invention proposes a method of modeling and simulating a biological system comprising one or more tangible biological functional entities modeled by at least:

a morphological occurrence comprising at least a biochemical constituent that identifies the persistent properties of the biological functional entity, and at least a transformation representing the way in which the constituent behaves as a function of the space-time context;

a spatial occurrence representing at least a spatial characteristic of the biological functional entity; and

a temporal occurrence representing at least a temporal characteristic of the biological functional entity;

which method simulates the behavior of said biological functional entities by recursively determining the effect on their functioning and on their behavior (their activities) of all changes affecting said occurrences, including transformations.

Thus the modeling and simulation method of the invention distinguishes between:

persistent characters (called “constituents” in the context of the invention) of the entities forming the biological system in question (name, category; for example: a cell, a nucleus, a protein, etc.); and

functional states of those constituents that depend on the active morphological occurrence of the entities and the space-time context in which they are active.

By means of the above features, the biological system to be simulated is modeled explicitly, in a standardizable and logical manner that is readily accessible to a biologist who is not a specialist in mathematics or programming (in particular, a biologist can readily verify the morphological characteristics and space-time characteristics, which are factual).

Moreover, the method of the invention separates simulation, which uses diverse mathematical calculation methods, and modeling: thus it is possible to apply a plurality of simulation methods to the same biological system model, either during successive simulations or simultaneously by applying a plurality of types of simulation to different portions of the biological system.

Various implementations of the method of the invention may have one or more of the following features, where appropriate:

the temporal occurrence is selected from an age of the biological functional entity and a period during which that functional entity is active;

said biological constituent is selected from an organism, a tissue, a cell, an organite, and a molecule;

said transformation is selected from a cellular transformation and a molecular transformation;

said transformation is a molecular transformation selected from:
a covalent molecular transformation, itself selected from a covalent transformation of proteins corresponding to a post-traductional transformation or a co-traductional transformation (such as a phosphorylation or a dephosphorylation), a covalent RNA transformation corresponding to RNA synthesis or maturation, and a covalent DNA transformation corresponding to DNA synthesis, damage or repair; and
a non-covalent transformation, itself selected from a hydrophobic transformation, a transformation caused by Van der Waals forces, electrostatic forces ox attraction between an electronegative atom of one molecule and a hydrogen atom of another molecule, and a steric transformation caused by attraction between adjacent atoms;

some of said function biological entities are included in a higher biological functional entity;

at least some of said biological functional entities include lower biological functional entities;

some functional entities constitute the environment of at least some other functional entities with which they interact;

the biological system further comprises intangible biological functional entities modeled by temporal occurrences and where appropriate by spatial and morphological occurrences;

said intangible biological functional entities comprise biochemical reactions,

Furthermore, the invention also consists in a model intended in particular (but not exclusively) for implementing a method as defined above, the model comprising one or more tangible biological functional entities modeled by at least:

a morphological occurrence comprising at least a biochemical constituent that identifies the biological functional entity, and at least a transformation representing the way in which that constituent behaves as a function of the space-time context;

a spatial occurrence representing at least a spatial characteristic of the biological functional entity; and

a temporal occurrence representing at least a temporal characteristic of the biological functional entity.

### DETAILED DESCRIPTION OF A PREFERRED EMBODIMENT

Other features and advantages of the invention become apparent in the course of the description below of an implementation of the invention given by way of non-limiting example.

The invention proposes a metamodel that is particularly suitable for modeling biological systems, in particular with a view to simulation for estimating their behavior.

According to the invention, the biological system is modeled as a set of tangible or intangible biological functional entities.

Each tangible biological functional entity is modeled by at least:

a morphological occurrence including a biochemical constituent that identifies the biological functional entity and a transformation representing the way in which that constituent behaves as a function of the space-time context;

a spatial occurrence representing a spatial characteristic of the biological functional entity (in particular the age of that entity and/or a period of activity of that entity); and

a temporal occurrence representing a temporal characteristic of the biological functional entity (corresponding to the position of the biological functional entity relative to its external environment), for example.

Where appropriate, each tangible biological functional entity may be included in a higher biological functional entity and/or may itself include lower biological functional entities. Moreover, at least some functional entities may constitute the environment of at least some other functional entities with which they interact.

The biochemical constituents referred to above may comprise, for example:

a living organism;

a biological tissue;

a cell, for example an epithelial cell or a lymphocyte cell;

an organite, for example a nucleus or a ribosome; and

a molecule, for example an RNA molecule, a DNA element (for example a gene, a regulator element or a promoter), or a protein.

The transformations mentioned above may include cellular transformations and molecular transformations, for example.

The molecular transformations in question may comprise, for example:

covalent transformations of proteins (in particular post-traductional transformations, such as phosphorylation or dephosphorylation, and co-traductional transformations), covalent. RNA transformations (in particular synthesis and splicing), and covalent DNA transformations (in particular synthesis, damage and repair by base excision, recombination, excision of nucleotides, photoreactivation and repairing matches); and

non-covalent hydrophobic molecular transformations, or transformations arising from Van der Waals forces, electrostatic forces or attraction between an electronegative atom of one molecule and an hydrogen atom of another molecule, or steric transformations caused by attraction between adjacent atoms (in particular phospho-isomerization).

Moreover, at least some of the biological functional entities modeling the biological system may be intangible biological functional entities, in particular biochemical reactions.

These intangible biological functional entities need not include morphological occurrences, in which case they include only temporal occurrences and where applicable spatial occurrences.

The biological system referred to above may be modeled in particular using the UML™ language and modeled functional units such as UML™ active objects, which facilitates mapping to formal languages facilitating analysis and simulation, for example the Pi Calculus language.

The status of each biological functional entity is defined at all times by the time and space values of these morphological occurrences and the behavior of the biological system over time is represented by the trajectory of the states of each biological functional entity in the frame of reference (shape, time, space).

To determine this behavior, the behavior of the biological functional entities over time is simulated recursively, progressively determining if at least some of the morphological transformations of said biological functional entities are produced as a function of the space-time context, as and when the biological functional entities interact.

Thus carrying out an action, for example a transformation, changes the value of the shape occurrence and changes the state of the functional entity, which then effects a transition, for example from the inactive state to the active state; the production of this new state constitutes an event that can trigger another action, for example transfer of the functional entity from one cellular compartment to another by modifying the spatial occurrence value.

The metamodel of the invention may be used to model and simulate, for example:

biochemical reactions, in which case the morphological occurrences of the biological functional entities (for example enzymes) comprise the biochemical compounds that are the subject of these reactions; to simulate the behavior of populations of functional entities (for example enzymes), the “population” functional entities may comprise concentrations of the constituents, for example, the reactions to be simulated then being modeled in a “protocol” file or by the morphological characteristics of a higher biological functional entity, and the simulation being effected by solving differential equations, for example, as in the “E-cell” software, or by a stochastic algorithm, as in the “Stochsim” software;

any biological process, for example using the prior art “Genomic Object Net” simulation software.

1. A method of modeling and simulating a biological system comprising one or more tangible biological functional entities modeled by at least:
a morphological occurrence comprising at least a biochemical constituent that identifies the persistent properties of the biological functional entity, and at least a transformation representing the way in which that constituent behaves as a function of the space-time context;
a spatial occurrence representing at least a spatial characteristic of the biological functional entity; and
a temporal occurrence representing at least a temporal characteristic of the biological functional entity;
which method simulates the behavior of said biological functional entities by recursively determining the effect on their functioning and on their behavior (their activities) of all changes affecting said occurrences, including transformations.

2. A method according to claim 1, wherein the temporal occurrence is selected from an age of the biological functional entity and a period during which that functional entity is active.

3. A method according to claim 1 or claim 2, wherein said biological constituent is selected from an organism, a tissue, a cell, an organite, and a molecule.

4. A method according to any preceding claim, wherein said transformation is selected from a cellular transformation and a molecular transformation.

5. A method according to any preceding claim, wherein said transformation is a molecular transformation selected from:
a covalent molecular transformation, itself selected from a covalent transformation of proteins corresponding to a post-traductional transformation or a co-traductional transformation, a covalent RNA transformation corresponding to RNA synthesis or maturation, and a covalent DNA transformation corresponding to DNA synthesis, damage or repair; and
a non-covalent transformation itself selected from a hydrophobic transformation, a transformation caused by Van der Waals forces, electrostatic forces or attraction between an electronegative atom of one molecule and a hydrogen atom of another molecule, and a steric transformation caused by attraction between adjacent atoms.

6. A method according to any preceding claim, wherein some of said function biological entities are included in a higher biological functional entity.

7. A method according to any preceding claim, wherein at least some of said biological functional entities include lower biological functional entities.

8. A method according to any preceding claim, wherein at least some functional entities constitute the environment of at least some other functional entities with which they interact.

9. A method according to any preceding claim, wherein the biological system further comprises intangible biological functional entities modeled by temporal occurrences and where applicable spatial and morphological occurrences.

10. A method according to claim 9, wherein said intangible biological functional entities comprise biochemical reactions.

11. A model for implementing a method according to any preceding claim, the model comprising one or more tangible biological functional entities modeled by at least:
a morphological occurrence comprising at least a biochemical constituent that identifies the biological functional entity, and at least a transformation representing the way in which that constituent behaves as a function of the space-time context;
a spatial occurrence representing at least a spatial characteristic of the biological functional entity; and
a temporal occurrence representing at least a temporal characteristic of the biological functional entity.
